(19) United States
(12) Patent Application Publication
(10) Pub. No.: US 2016/0047789 A1
(43) Pub. Date: Feb. 18, 2016

(54) DETECTION OF TRACE POLAR COMPOUNDS BY OPTICAL SENSORS

(71) Applicant: UNIVERSITY OF TENNESSEE RESEARCH FOUNDATION, Knoxville, TN (US)

(72) Inventors: Ziling Xue, Knoxville, TN (US); Jonathan Fong, Knoxville, TN (US)

(21) Appl. No.: 14/776,456

(22) PCT Filed: Mar. 13, 2014
(86) PCT No.: PCT/US14/26059

§ 371 Date: Sep. 14, 2015

Related U.S. Application Data
(60) Provisional application No. 61/780,884, filed on Mar. 13, 2013.
(60) Provisional application No. 61/857,592, filed on Jul. 23, 2013.

(51) Int. Cl. G01N 33/22; G01N 21/78

(52) U.S. Cl. CPC G01N 33/225; G01N 21/78; G01N 2021/7769

(57) ABSTRACT

A method of detecting a polar compound in a sample, such as a fuel sample. The method including: contacting the sample with a solvatochromic compound, wherein the solvatochromic compound undergoes a shift in spectral absorbance when in contact with polar compounds in the sample; and detecting a change in spectral absorbance of the solvatochromic compound, thereby detecting polar compounds in the sample.

### CROSS-REFERENCE TO RELATED APPLICATIONS

This application claims the priority benefit of U.S. Provisional Application No. 61/780,884, filed on Mar. 13, 2013 and U.S. Provisional Application No. 61/857,592, filed on Jul. 23, 2013, both of which are incorporated herein by reference in their entirety.

### FIELD

This disclosure relates chemical analysis, and specifically to the detection of polar compounds in fuels with solvatochromic compounds.

### BACKGROUND

Biofuels have become an area of intense interest in recent years. With the rapid depletion of non-renewable fossil fuels, there is an increasing demand for alternative energy sources. Thus, a greater emphasis has been put on developing and producing renewable forms of energy such as biofuels and in particular cellulose based biofuels and biodiesel (see e.g. Sharma et al. Fuel 87, 2355-2373 (2008)).

Several key advantages of biofuel usage are the decreases in the emissions of hydrocarbons, carbon monoxide, and sulfur dioxide as well as their carbon neutral. Unfortunately, adulteration of biofuels with contaminants is a major concern and may lead to slower than desirable adoption of these alternative fuel sources.

Cellulose biofuels generated by pyrolysis of biomass such as tree barks and leaves are an active area of study as a green energy source. One major hurdle in the adoption of cellulose biofuels is their undesirable high oxygen content and high acidity, even after hydrotreatment. This oxygen content is primarily from phenols and carboxylic acids generated from the degradation of hemicelluloses. The presence of these highly polar compounds makes the fuel unstable and insoluble or immiscible in hydrocarbons such as petroleum.

Phenols and N—H bond containing compounds, such as indoles and carbazoles, are trace (<0.1% by mass) polar species in jet fuel that affect fuel thermal and storage stability, combustion efficiency, or combustion emissions. Analysis of these polar compounds is challenging, as jet fuel is a complex mixture with hundreds of species as different groups of chemicals. Each major group contains alkyl derivatives of the parent compounds with similar chemical properties.

Generally, biodiesel consists of fatty acid methyl esters (FAME). FAME is usually produced by a transesterification process from vegetable oil, animal fat, or cooking grease where triglycerides are cleaved and reacted with methanol to produce glycerol and FAME. With the passage of ASTM D975 and EN 590 biodiesel regulations, the U.S allows on-and-off-road diesel to contain up to 5% v/v FAME while European countries permit up to 7% v/v FAME in automotive diesel without specific labeling (see for example, ASTM International D975-09. Standard Specification for Diesel Fuel Oils; European Standard EN 14214:2008+A1:2009 Automotive fuels. Fatty acid methyl esters (FAME) for diesel engines. Requirements and test methods; Nygren et al. Energy Policy 37, 4003-4010 (2009); and Froment FAST 46, (2010)). In most cases, fuel distribution and supply systems use the same refineries, storage tank facilities, and pipelines for the majority of transportation fuels. This practice raises a difficult problem regarding cross fuel contamination because FAME is a highly surface active material, adhering to pipelines and distribution tank walls during its transportation. Therefore, FAME can potentially desorb into other transportation fuels that use the same pipelines or storage tanks.

In the aviation industry, there is a particular concern over FAME contamination in jet fuel because at higher levels it can impact the thermal stability and freezing point of jet fuel leading to deposits in the fuel system or cause the fuel to gel. These issues can result in jet engine operability problems and possible engine flameout. Currently, the aviation industry allows up to 5 ppm FAME in its jet fuel before it is off specification (Pauls Journal of Chromatographic Science 49, 384-396 (2011)). While protective measures and strict regulations are in place to prevent cross contamination, the permitted threshold of FAME in jet fuel makes it difficult to completely prevent FAME contamination in a shared fuel system (Rintoul Biodiesel Magazine, 10 Sep. 2012). Since 5 ppm FAME in jet fuel has proved to be difficult to detect, very sophisticated instrumentation is needed in order to determine FAME contamination.

Thus, there is a need to develop quick, easy, and direct methods for the detection of polar compounds in amongst other applications in detecting fuel contaminants, for example FAME, in non-polar liquids such as fuel. For example rapid FAME detection would be an indispensable tool since current FAME detection methods are more amenable to laboratory testing where waiting for an off-site test may take several hours to days. Similarly, the there is a need to detect polar compounds in aviation fuel, such as phenols, indoles and the like. There is also a need to detect highly concentrated FAME in fuels such as 20% biodiesel in diesel (B20) as quick verification check of the biodiesel blend accuracy.

### SUMMARY

Disclosed is a method of detecting a polar compound in a sample, the method including contacting the sample with a solvatochromic compound (such as Nile Blue Chloride (5-amino-9-(diethylamino)benzo[a]phenoxazin-7-ium chloride or NBC), brilliant cresyl blue, resorufin (7-Hydroxy-3H-phenoxazin-3-one 10-oxide), or rhodamine 6g (r6g), wherein the solvatochromic compound undergoes a shift in spectral absorbance when in contact with the polar compounds in the sample as compared to the absorbance of the solvatochromic compound in a non-polar solvent; and detecting a change in spectral absorbance of the solvatochromic compound, thereby detecting the polar compound in the sample. Examples of polar compounds that can be detected with the disclosed method include without limitation, esters (such as fatty acid methyl esters), alcohols (such as phenols), carboxylic acids, an amines (such as anilines, pyrroles, indoles, carbazoles, pryrimidines, and the like), amides, or any combination thereof. In particular examples, the disclosed method is a method of detecting a fatty acid methyl ester (FAME) in fuel sample, such as a diesel sample or jet fuel sample this contaminated or is suspected of being contaminated with FAME, for example FAME arising from biodiesel production.

In some embodiments, the detected absorbance is compared to a control, for example for qualitative or quantitative detection of polar compounds in a sample. Examples of controls of use in the disclosed method include values indicative of a known concentration of a known polar compound, one of more control samples with a known concentration of a known polar compound, a spectral absorbance of one of more control samples with a known concentration of a known polar compound, a calibration curve for a known polar compound or any combination thereof. In some examples, the detection of a change in spectral absorbance is made using UV/vis spectrophotometry. In some examples, the detection of a change in spectral absorbance is made by visual inspection, for example by observation of a color change in the sensor by an individual.

In some embodiments, the solvatochromic compound is immobilized in or on a solid or semi solid material, such as in a film or membrane, for example as a sensor or part of a sensor array. Examples of solid or semi solid material of use for making sensors include ethyl cellulose, polymers, silica sol-gels, or organically modified sol-gels amongst others.

### BRIEF DESCRIPTION OF THE DRAWINGS

FIG. 1 is a schematic and a set of digital images showing how the disclosed polar compound sensors change color when exposed to a polar compound such as FAME. While particular emphasis is given to the detection of FAME, the methods and compositions disclosed herein are equally applicable to the detection of other polar compounds.

FIGS. 2A and 2B are digital images of scanning electron microscope (SEM) surface images of the thin film sensors before (FIG. 2A) and after (FIG. 2B) exposure to FAME. The particles in FIG. 2B are due to the sputtered gold particles needed on the sensor's surface to increase the conductivity for SEM imaging.

FIG. 3 is an absorbance spectrum of an exemplary sensor showing the formation of a new peak at 500 nm upon exposure of the solvatochamatic sensor to a polar compound.

FIG. 4 is a calibration plot and confirmation using biodiesel samples in the 0.5-30 ppm range.

FIGS. 5A and 5B are curves showing the detection of FAME in 0-5% v/v FAME mixtures modeled by a logarithmic curve function (A) raw (B) linearized.

FIG. 6 is a set of curves showing the absorbance of NBC when dissolved in MeOH (625 nm) and FAME (525 nm). This result demonstrates that NBC can be used as a sensor of polar compounds such as FAME.

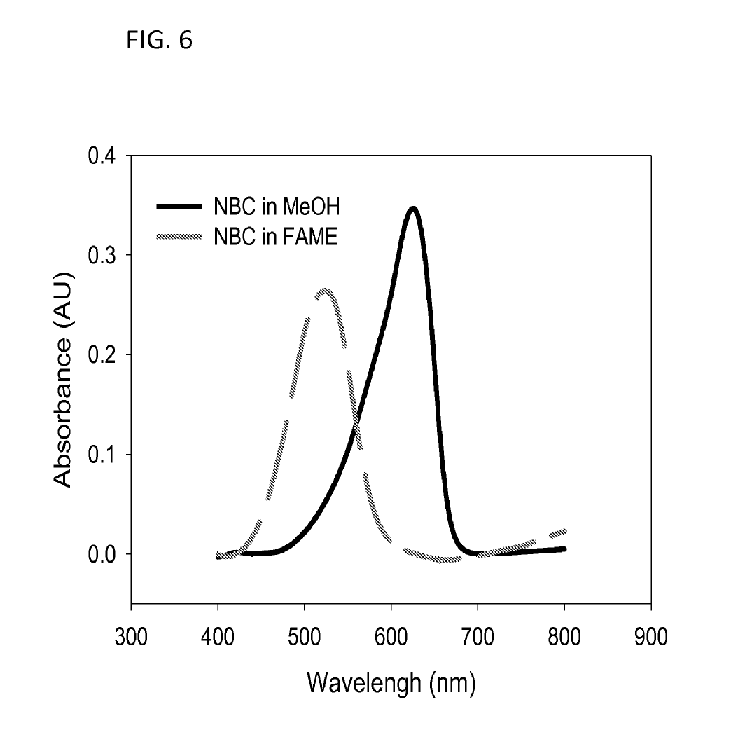

FIGS. 7A and 7B are calibration plots. FIG. 7A, calibration plot of the sensor demonstrating the detection of FAME from 0-10 ppm. FIG. 7B, absorbance spectra showing the peak at 500 nm increasing with increasing concentrations of FAME mixture (0-0.001% v/v).

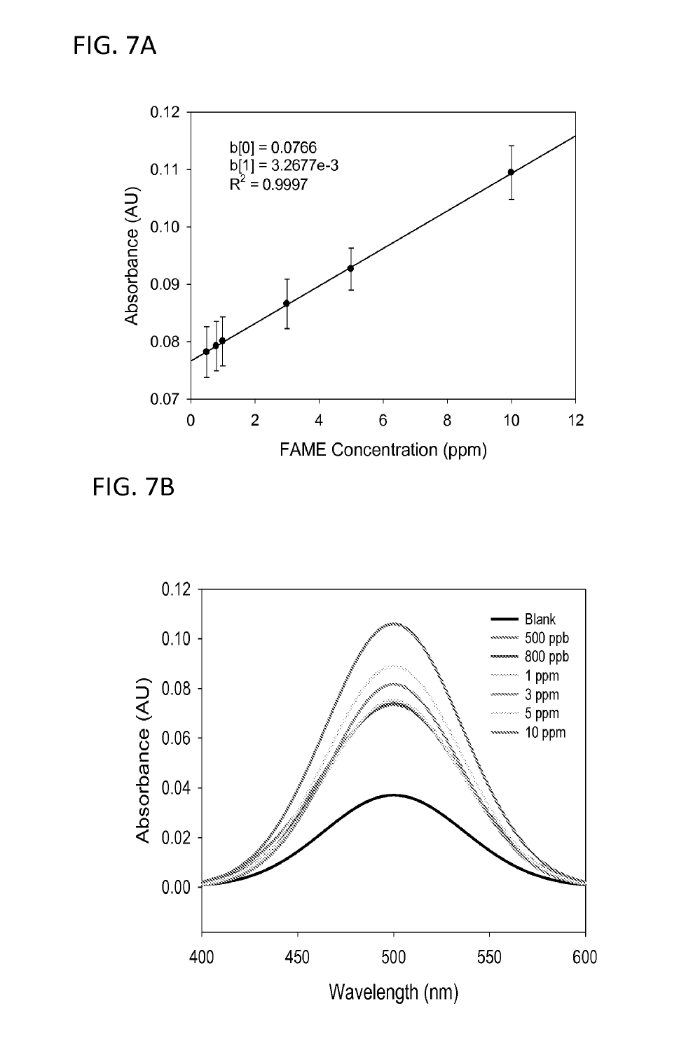

FIGS. 8A and 8B is (A) the structure of Brilliant Cresyl Blue (BCB) and (B) digital images of BCB dissolved in methanol (left) and FAME (right).

FIGS. 9A and 9B is a (A) digital image of a comparison of BCB film with no FAME exposure (left) and after FAME exposure (right) and (B) UV-Vis spectra of BCB film exposed to kerosene and FAME.

FIGS. 10A and 10B is (A) the structure of Resorufin and (B) digital images of resorufin dissolved in methanol (left) and FAME (right).

FIGS. 11A and 11B is (A) a digital image of resorufin thin film without exposure to FAME (left) and exposed to FAME and (B) UV-Vis spectra of Resorufin thin film exposed to kerosene and FAME.

FIGS. 12A and 12B is (A) the structure of Rhodamine 6G (R6G) and (B) a digital image of R6G dissolved in methanol (left) and FAME (right).

FIGS. 13A and 13B is (A) a digital image comparing R6G films without exposure to FAME (left) and exposure to FAME (right) and (B) UV-Vis spectra of R6G exposed to kerosene and FAME.

FIGS. 14A and 14B is (A) the structure of Ethidium Bromide and (B) a digital image of Ethidium Bromide dissolved in methanol (left) and FAME (right).

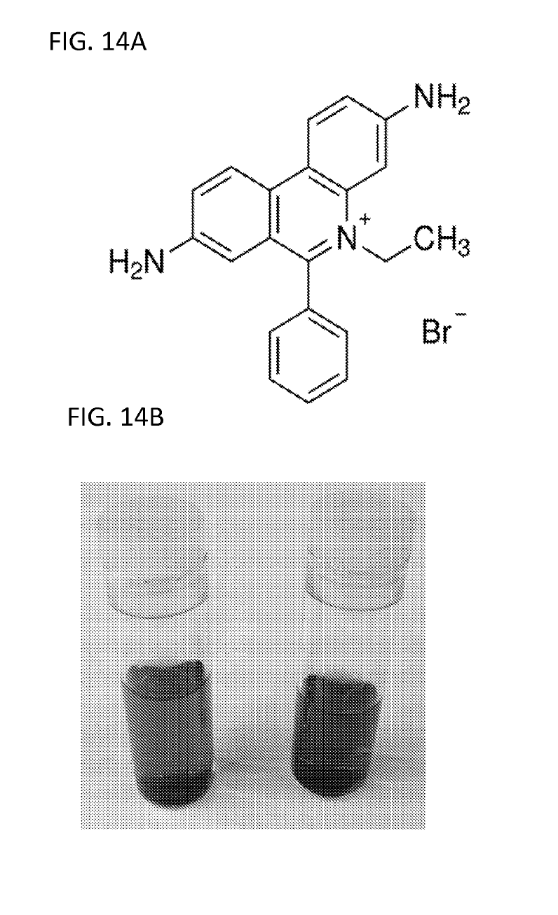

FIGS. 15A and 15B is (A) a digital image of a comparison of Ethidium bromide films without exposure to FAME (left) and after exposure to FAME (right) and (B) UV-Vis spectra of Ethidium bromide exposed to kerosene and FAME.

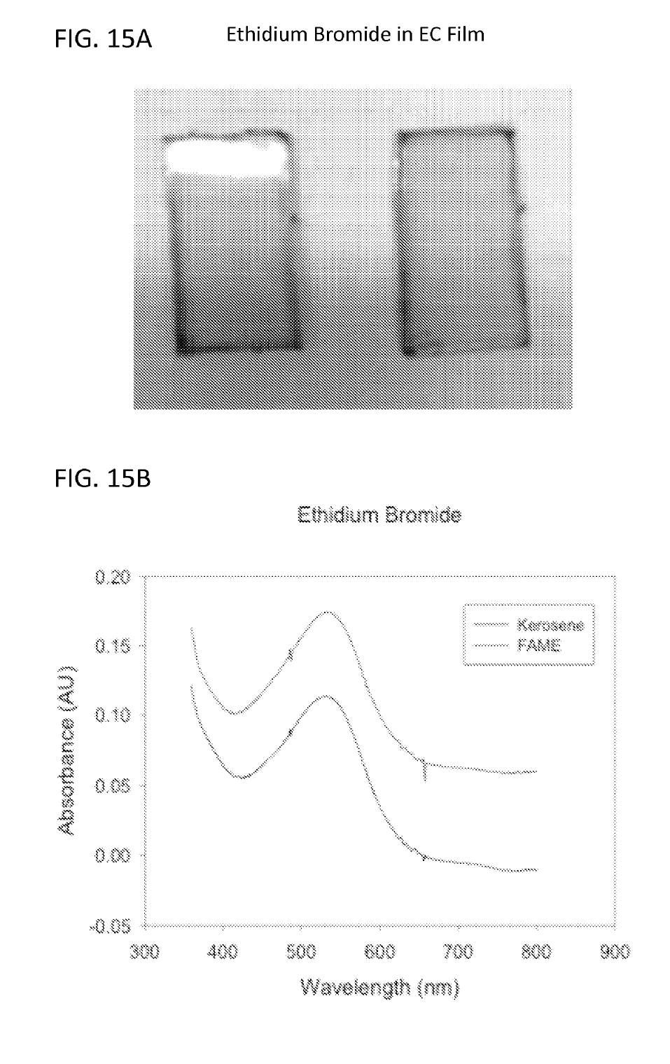

FIGS. 16A and 16B is a set of curves showing the detection of FAME in real world samples of biodiesel modeled by a logarithmic curve function (A) raw (B) linearized.

FIG. 17 is a set of digital images of sensors after exposure to PhOH solutions.

FIG. 18 is a set of visible spectra of sensors after exposure to PhOH solutions in kerosene

FIG. 19 is a calibration curve for the sensor response to PhOH solutions.

FIG. 20 is a set of digital images of (Top) sensors before (left) and after (right) exposure to pure PhNH2 (aniline) and (Bottom) Spectra of the two sensors.

FIG. 21 is a set of digital images of sensors after exposure to carbazole solutions.

FIG. 22 is a set of digital images of sensors after exposure to pyrrole solution.

### DETAILED DESCRIPTION

### I. Summary of Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Unless otherwise noted, technical terms are used according to conventional usage. Definitions of common terms in chemistry may be found in Richard J. Lewis, Sr. (ed.), Hawley's Condensed Chemical Dictionary, published by John Wiley & Sons, Inc., 1997 (ISBN 0-471-29205-2).

To facilitate review of the various embodiments of the disclosure, the following explanations of specific terms are provided:

Binding: A solvatochromic compound (dye) binds or stably binds to a target, such as a polar compound, for example a fatty acid methyl ester (FAME). Binding can be detected by either physical or functional properties, for example a change in the abosorbance and/or emission spectrum of the solvatochromic compound.

Contacting: Placement in direct physical association including both in solid or liquid form, for example contacting a sample, such as a sample believed to contain or being tested for the presence of a polar compound, such as FAME, with a solvatochromic compound.

Detect: To determine if an agent (such as a signal or a polar compound, such as FAME) is present or absent. In some examples, this can further include quantification.

Fatty acid methyl ester: A type of fatty acid ester that can be produced by an alkali-catalyzed reaction between fats or fatty acids and methanol. The molecules in biodiesel are primarily FAME, usually obtained from vegetable oils by transesterification,

Polar compound: A compound in which the electric charge is not symmetrically distributed, so that there is a separation of charge or partial charge and formation of definite positive and negative poles. Examples of polar compounds include esters, alcohols, amines and other related compounds.

Solvatochromic compound: A compound, such as a dye that changes color due to a change in solvent polarity.

Solvatochromism: The change in position and sometimes intensity of a UV-visible absorption band following a change in the polarity of the solvent.

Suitable methods and materials for the practice or testing of this disclosure are described below. Such methods and materials are illustrative only and are not intended to be limiting. Other methods and materials similar or equivalent to those described herein can be used.

### II. Description of Exemplary Embodiments

### A. Introduction

Typical methods to analyze the polar species in fuel samples, such as aviation fuels, require the use of centralized and expensive instrument such as gas chromatography-mass spectrometry (GC-MS), GC-a pulsed flame photometric detector (GC-PFPD) and ammonia chemical ionization MS, particle beam liquid chromatography/MS (LC-MS), MS with atmospheric pressure chemical ionization, positive-ion electrospray ionization MS (ESI), time-of-flight secondary ion MS (TOF-SIMS) and electron ionization, field desorption ionization, and electrospray ionization Fourier transform ion cyclotron resonance MS (FT-ICR MS), (see for example Lissitsyna et al, Chromatographia 75, 1319-1325 (2012); Pardo et al. J. Am Oil Chem Soc 89, 631-637 (2012); Seeley et al. Journal of Chromatography A 1226, 103-109 (2012); Tariq, et al. Fuel Process. Technol. 92, 336-341 (2011); Kaminski et al. J. Chromatogr. A 1122, (2006) 153-160; Carvalho et al. Journal of the Brazilian Chemical Society 23, 763-769 (2012); Mello et al. Magnetic Resonance in Chemistry 46, 1051-1054 (2008); Monteiro et al. Fuel 88, 691-696 (2009)). Despite the regular use of these standard techniques, there are noticeable drawbacks such as bulky and expensive instrumentation, and portability issues. There are hundreds of species in aviation fuels and it is difficult and challenging to analyze polar species by GC-MS. The detection by infrared cannot reach the 5 ppm detection limit.

To solve the aforementioned problems, the inventors have developed a set of UV-visible optical sensors employing solvatochromatic dyes and a method of detecting polar compounds using the same. The disclosed sensors are intrinsically easy to use, inexpensive, and can be mass-produced for disposable applications. Moreover, the combination of dye-doped optical sensors and spectrophotometers makes remote sensing possible.

As disclosed herein, solvatochromatic dyes, and in particular examples Nile Blue Chloride (NBC), were incorporated into a thin ethyl cellulose film as an optical sensor for the detection of polar compounds, and in certain applications for use in FAME detection.

NBC, an oxazine dye, contains two amino groups, which form a push-pull conjugated system with a heterocyclic oxygen atom to bridge the conjugation. An oxazine dye such as NBC displays positive or negative solvatochromism with a large dipole moment change when its solvent becomes more polar (Jose et al. Chemistry—A European Journal 15, 418-423 (2008); Peng et al. Spectrochimica Acta Part A: Molecular and Biomolecular Spectroscopy 75, 1571-1576 (2010)).

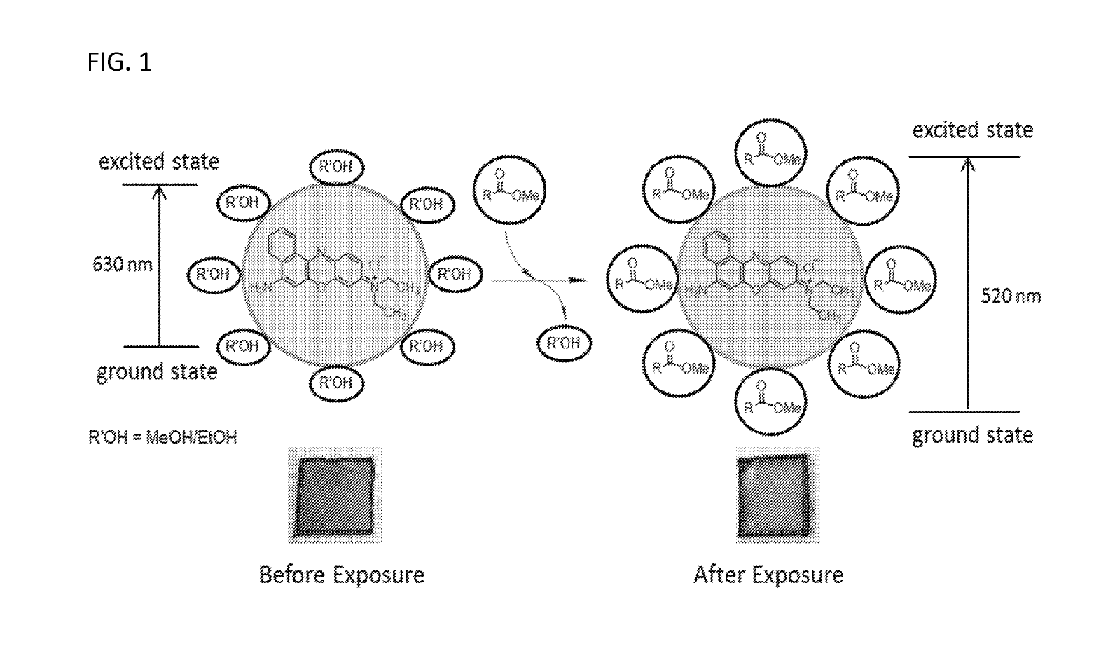

With reference to the detection of FAME, the inventors prepared a sensor film containing Nile Blue Chloride (NBC) in methanol/ethanol, which displays a blue color. In FAME-containing diesel, FAME replaces the alcohols, changing the sensor color to pink (FIG. 1). The color change may be monitored by naked eye or by a visible spectrometer. The sensor has been successfully used for quantitative FAME detection in the 0.5-200,000 ppm (20% v/v) range. As detailed in the Examples below, this technique was applied to other solvatochromic compounds. Furthermore, as disclosed in the Examples below, NBC used in the detection biodiesel/FAME, has been applied to an optical sensor for the detection of polar species in both phenols and N—H containing polar species in kerosene (as simulated jet fuel).

The results presented herein demonstrate that the disclosed sensors are ideal for the detection of polar compounds, such as those that may be present in fuel samples. The disclosed sensors and methods of use offer an inexpensive alternative to other techniques available and are usable with little training.

### B. Methods of Detection

The ability to detect polar compounds in fuel samples, such as fatty acid methyl esters (FAME or biodiesel) at low concentrations in diesel or aviation fuel is an important goal in many industrial sectors. Disclosed herein is a simple, optical sensor containing a solvatochromatic compound, such as Nile Blue Chloride (NBC), for quick and direct detection of polar compounds, for example FAME detection in fuel samples. The sensing mechanism of the sensor relies on the solvatochromatic properties of the compound, which undergoes a color change in the presence of a polar compound, such as FAME, relative to solvation in a non-polar solvent, such as a fuel sample. The disclosed method of detecting a polar compound in a sample includes contacting the sample with a solvatochromic compound, wherein the solvatochromic compound undergoes a shift in spectral absorbance when in contact with the polar compound in the sample as compared to the absorbance of the solvatochromic compound in a another solvent, such as a non-polar solvent. The spectral absorbance and/or emission is detected. A change in the spectral absorbance, for example relative to a control, indicates the presence of polar compounds in the sample, such is a fuel sample, for example a fuel sample suspected of containing a polar compound or a fuel sample being tested for the presence of a polar compound.

In some embodiments, the method is used to detect esters, alcohols, carboxylic acids, amines, amides, or any combination thereof. In specific examples the polar compound comprises a fatty acid methyl ester (FAME), for example a fatty acid methyl ester from biofuel, such as biodiesel. In some examples, the method is used to detect an alcohol such as a phenol. In some examples, the method is used to detect and amine such an aniline, pyrrole, indole, carbazole, prymadine, or a combination thereof. In some examples, the method is used to detect an ester, an alcohol, a carboxylic acid, an amine, an amide, a pyrrole, an indole, a carbazole, a pyridine, a quinolone, a sulfur-containing compound such as a thiol, a thioether, a thiophenol, a ketone, an aldehyde, an ether, an acyl halide, an alkyl halide, cyclothiazide, 1,2-dithiane, benzothiazole, 2-benzothiophene, dihydro-5,6-dimethyl for a combination thereof

In some embodiments, the polar compound is a product of pyrolysis of biomass, for example the product of the pyrolysis of cellulose, lignin or a combination thereof and the like.

Thus, disclosed is a method of detecting fatty acid methyl esters in a sample, such as a fuel samples, for example a sample of diesel fuel. The method involves contacting the sample with a solvatochromic compound that undergoes a shift in spectral absorbance when in contact with the fatty acid methyl esters in the sample.

Upon contact with the sample, optical responses, or spectral absorbance, can be detected, which in some examples are recorded. The optical response generated can be intensity changes, spectral shift, and time-dependent variations associated with the sensor elements upon exposure, for example to a sample or a reference fluid (such as methanol, kerosene ethanol, DMF, DCM, acetone, acetic acid, toluene, etc.). In some examples, a light source, for example multicrhomatic light source is used as an excitation source for the solvatochromic compound. The light can be filtered by an excitation filter before reaching the sample. Spectral absorbance can be detected by any methods known in the art, for example using a spectrophotometer.

Solvatochromic compounds are compounds having spectroscopic characteristics (e.g., absorption, emission, fluorescence, phosphorescence) in the ultraviolet/visible/near-infrared spectrum that are influenced by the surrounding medium. Both the wavelength-dependence and the intensity of a compound's spectroscopic characteristics are typically affected. Examples of solvatochromic compound suitable for use with the disclosed methods include any known solvatochromic compound. Solvatochromic compounds have been extensively reviewed in, for example, Reichardt, Chemical Reviews, 94:2319-2358 (1994); Reichardt et al. Pure and Applied Chemistry 65(12):2593-601 (1993); and Buncel and Rajagopal, Accounts of Chemical Research, 23(7):226-31 (1990), all of which are incorporated herein be reference in their entirety.

Characteristics of solvatochromic compounds include positive or negative solvatochromic, which corresponds to the bathochromic and hypsochromic shifts, respectively of the emission band with increasing solvent polarity. In addition to the solvent-induced spectral shifts of the spectral absorbance, some compounds exhibit the solvent-dependent ratio of emission intensities of two fluorescence bands. One such solvatochromic compound is pyrene (1-pyrenebutanoic acid). Examples solvatochromic compounds include, but are not limited to 4-dicyanmethylene-2-methyl-6-(p-dimethylaminostyryl)-4H-pyran (DCM); 6-propionyl-2-(dimethylamino)naphthalene (PRODAN); 9-(diethylamino)-5H-benzo[a]phenoxazin-5-one (Nile Red); 4-(dicyanovinyl)julolidine (DCVJ); phenol blue; stilbazolium dyes; coumarin dyes; ketocyanine dyes; Reichardt's dyes including Reichardt's Betaine dye (2,6-diphenyl-4-(2,4,6-triphenylpyridinio) phenolate); merocyanine dyes, including merocyanine 540; N,N-dimethyl-4-nitroaniline (NDMNA) and N-methyl-2-nitroaniline (NM2NA); and the like. Other solvatochromic dyes include, but are not limited to Nile Blue Chloride; 1-anilinonaphthalene-8-sulfonic acid (1,8-ANS), and dapoxylbutylsulfonamide (DBS) as well as other dapoxyl analogs. In some embodiments, the solvatochromic dye comprises a lipophilic solvatochromic dye, for example one or more of Nile Blue (5-amino-9-(diethylamino)benzo[a]phenoxazin-7-ium chloride), brilliant cresyl blue, resorufin (7-Hydroxy-3H-phenoxazin-3-one 10-oxide), rhodamine 6g (r6g) or ethidium bromide.

In some embodiments, an appropriate solvent is used to dissolve the solvatochromic compound, one of ordinary skill in the art can choose an appropriate solvent without undue experimentation. In some UV-visible spectrum of the solution containing the solvatochromic compound in the solvent shows a change in absobance, such as one or more of absorptions at wavelengths different from the solution of the solvatochromic compound in a solution containing a polar compound (i.e. a color change), or different intensities at the same wavelength.

In the case of FAME detection, the highly sensitive, disposable sensors disclosed herein are capable of detecting 0.5-200,000 ppm (20% v/v) FAME in diesel. In certain embodiments, a detection range from about 20% down to 0.5 ppm biodiesel in diesel level is achievable. For example, the methods disclosed herein can detect FAME at a concentration of as low as about 200,000 ppm to about 5 ppm, such as about 150,000 ppm, about 125,000 ppm, about 100,000 ppm, about 80,000 ppm, about 60,000 ppm, about 50,000 ppm, about 40, 000 ppm, about 30,000 ppm, about 20,000 ppm, about 10,000 ppm, about 5,000 ppm, about 1,000 ppm, about 500 ppm, about 250 ppm, about 100 ppm, about 50 ppm, about 25 ppm, about 10 ppm or about 5 ppm, such as between about 200,000 ppm and 5 ppm, 100,000 ppm and 50,000 ppm, 150,000 ppm and 25,000 ppm, 20,000 ppm and 500 ppm, 10,000 ppm and 5 ppm and the like.

The solvatochromic compound can be immobilized in or on a solid or semi solid material (for example a porous material), such as a polymer. In specific examples, the solid or semi solid material comprises ethyl cellulose, polymer, silica sol-gels, or organically modified sol-gels. In certain example embodiments, the polymer and compound are fabricated to form a membrane or thin film, which can be adhered or otherwise physically joined to a substrate, which can provide physical support, for example as part of a test strip. Any material can be used so long is it is compatible with the film or membrane. Alternatively, the solvatochromic compound can be a solution or solid, such as a powder, and added directly to a liquid sample. In some examples, the solid or semi solid material comprises a membrane or thin film. In some examples, the solvatochromic compound is covalently attached the solid or semisolid surface, which can potentially be any size and/or shape. In some examples, the solvatochromic compound is non-covalently attached to the surface, for example adsorbed or otherwise bound. By “covalently attached” herein is meant that two moieties are attached by at least one bond, including sigma bonds, pi bonds and coordination bonds. The covalent attachment may be done using a linker, which has covalently attached solvatochromic compound and is itself covalently attached to the surface. Alternatively, there are a variety of entrapment systems that are used to entrap or contain compounds and other materials on our within a solid or semisolid surface.

The functionalization of solid support surfaces such as certain polymers with chemically reactive groups such as silanes, thiols, amines, carboxyls, etc. is generally known in the art. Some examples of these surface chemistries for blank microspheres include, but are not limited to, amino groups including silanes, hydroxy groups for silane attachment, aliphatic and aromatic amines, carboxylic acids, aldehydes, amides, chloromethyl groups, hydrazide, hydroxyl groups, sulfonates and sulfates.

These functional groups can be used to add any number of different moieties to the solid or semi-solid surface, generally using known chemistries. For example, linkers as outlined below comprising carbohydrates (e.g. polydextrans, etc.) may be attached to an amino-functionalized support; the aldehyde of the carbohydrate is made using standard techniques, and then the aldehyde is reacted with an amino group on the surface. In an alternative embodiment, a sulfhydryl linker may be used. There are a number of sulfhydryl reactive linkers known in the art such as SPDP, maleimides, α-haloacetyls, and pyridyl disulfides (see for example the 1994 Pierce Chemical Company catalog, technical section on cross-linkers, pages 155-200, incorporated herein by reference) which can be used to attach cysteine containing moieties (e.g. amino acid oligomers) to the support. Alternatively, an amino group on the compound may be used for attachment to an amino group on the surface. For example, a large number of stable bifunctional groups are well known in the art, including homobifunctional and heterobifunctional linkers (see Pierce Catalog and Handbook, pages 155-200). In an additional embodiment, carboxyl groups (either from the surface or from the candidate agent) may be derivatized using well known linkers (see the Pierce catalog). For example, carbodiimides activate carboxyl groups for attack by good nucleophiles such as amines (see Torchilin et al., Critical Rev. Therapeutic Drug Carrier Systems, 7(4):275-308 (1991), expressly incorporated herein). Preferred methods of attachment are shown in the figures, and utilize silane chemistry for the covalent attachment of compounds.

It should be understood that when the solvatochromic compounds are attached to the surface that the manner of attachment does not significantly alter the functionality of the compound; that is, the compound should remain solvatochromic.

In some embodiments, the spectral absorbance is compared to a control. In some embodiments, controls for use in the disclosed methods include one or more values indicative of a known concentration of a polar compound in a control sample, for example as a calibration curve. In some embodiments, controls for use in the disclosed methods include one or more values indicative of a known concentration of a polar compound in a control sample, for example as a calibration curve. In some examples the control is one of more control samples with a known concentration of polar compound or values derived therefrom, for example as a calibration curve. In some embodiments, the control is a calibration curve. In some embodiments, controls for use in the disclosed methods include one or more values indicative of a known concentration of fatty acid methyl in a control sample, for example as a calibration curve. In some examples the control is one of more control samples with a known concentration of fatty acid methyl esters or values derived therefrom, for example as a calibration curve. In some embodiments, the control is a calibration curve. Examples of polar compounds for use as a control include an alcohol, a carboxylic acid, an amine, an amide, a pyrrole, an indole, a carbazole, a pyridine, a quinolone, a sulfur-containing compound such as a thiol, a thioether, a thiophenol, a ketone, an aldehyde, an ether, an acyl halide, an alkyl halide, or a combination thereof.

In some embodiments, the sample comprises a fuel sample, such as a diesel fuel sample, a biofuel sample or an aviation fuel sample. In some examples, the biofuel sample comprises a pyrolysis product of biomass, such as a pyrolysis product of cellulose, lignin or a combination thereof.

In some embodiments, the method detects the amount of biodiesel present in a diesel fuel sample. In some embodiments, the method detects the amount of polar compounds present in a biofuel sample. In some embodiments, the method detects the amount of fatty acid methyl esters from biofuel, such biodiesel, for example as a contaminant in non-biodiesel diesel.

In certain embodiments, the method comprises an optical absorption technique. Additionally or alternatively, the method comprises a fluorescent technique using a compound such as Nile Blue Chloride. In certain examples, the detection can include detection with a spectrophotometer in the ultraviolet visible wavelength range. In some examples, the detected spectral absorbance is used for quantification of polar compounds in the sample. In some embodiments, the method further includes quantifying the amount of polar compounds in the sample. A sensor may be used to determine, with the naked eye, for example, detection and quantification of polar compounds in a fuel sample.

Aspects of the forgoing are illustrated by the following non-limiting examples.

### EXAMPLES

### Example 1

This example describes testing of solvatochromatic compounds to demonstrate their ability to detect polar compounds, and in particular to this example FAME in a sample.

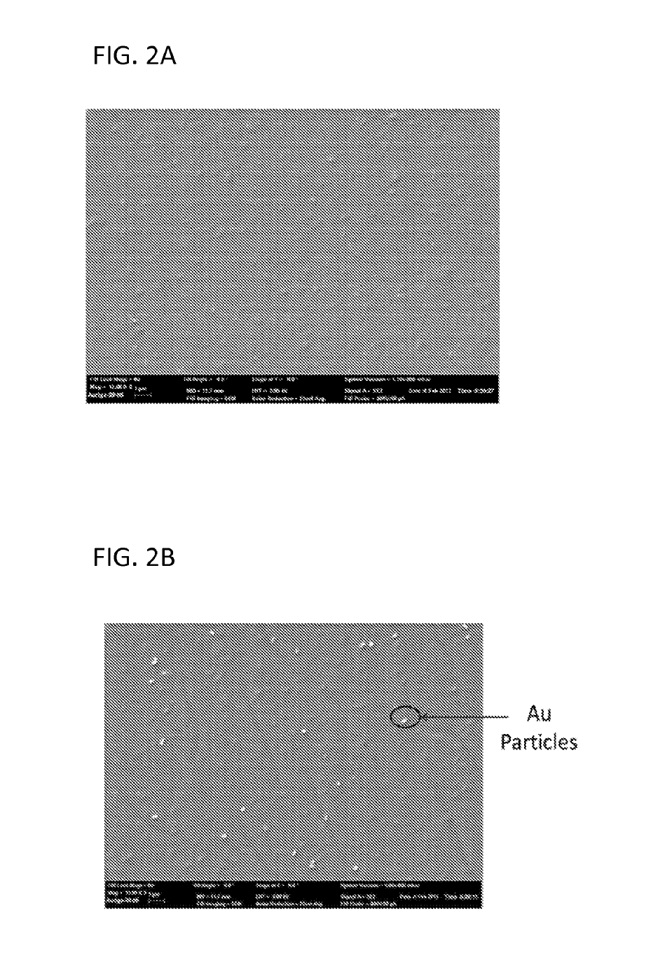

As disclosed herein, several solvatochromatic compounds were tested in various solutions of kerosene, MeOH, and FAME. Initial solution tests demonstrated that NBC is insoluble in nonpolar solutions such as diesel, turns to a blue color in MeOH with an absorbance at ˜625 nm, and changes to a pink color in a mixture of several different FAME solutions with an absorbance at ˜525 nm. Sensor fabrication delivered an effective process to encapsulate Nile Blue Chloride (NBC) in a polymer cellulose matrix resulting in distinct blue colored thin films. These blue thin films have an absorbance at 610 nm exhibiting a small blue shift in absorbance relative to the solution. Upon exposure of these sensors to a nonpolar solution (e.g., diesel) no color change, dye leaching, or an absorbance shift was observed. This result reinforced the choice of using the polymer cellulose matrix for the fabrication of a sensor, although other material can also work. Scanning Electron Microscope (SEM) imaging was taken in order to characterize the surface of the FAME sensor before and after exposure to the FAME mixture. The images show features “porous pitting” consistent with the phase separation that occurs during the processing of the sensor with no discernible difference on the surface of the sensor before or after exposure (see FIG. 2). The porosity and polar features of the cellulose film allow FAME to preconcentrate and diffuse into the sensor, causing a color change and blue shift upon exposure of the sensor to FAME.

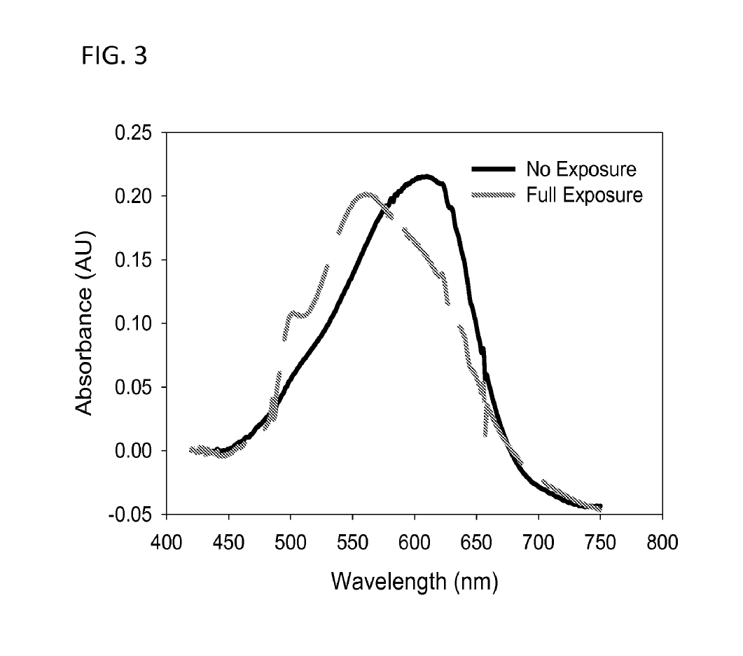

A distinct color change (blue to pink) was observed when the sensor is exposed to FAME, a less polar solution, with a blue shift in absorbance to 560 nm (FIG. 1). While not being bound by theory, this color change is believed to be largely attributed to FAME molecules replacing alcohols that surround the sensor during its fabrication, as a result of the positive solvatochromatic property of NBC. The spectrum of the sensor shows a peak at 500 nm that gives the sensor pink color (FIG. 3). These results demonstrate that the disclosed sensors provide a quick, direct, and disposable method for FAME determination that is unlike any other standard detection method in the field such as GC-MS or FT-IR.

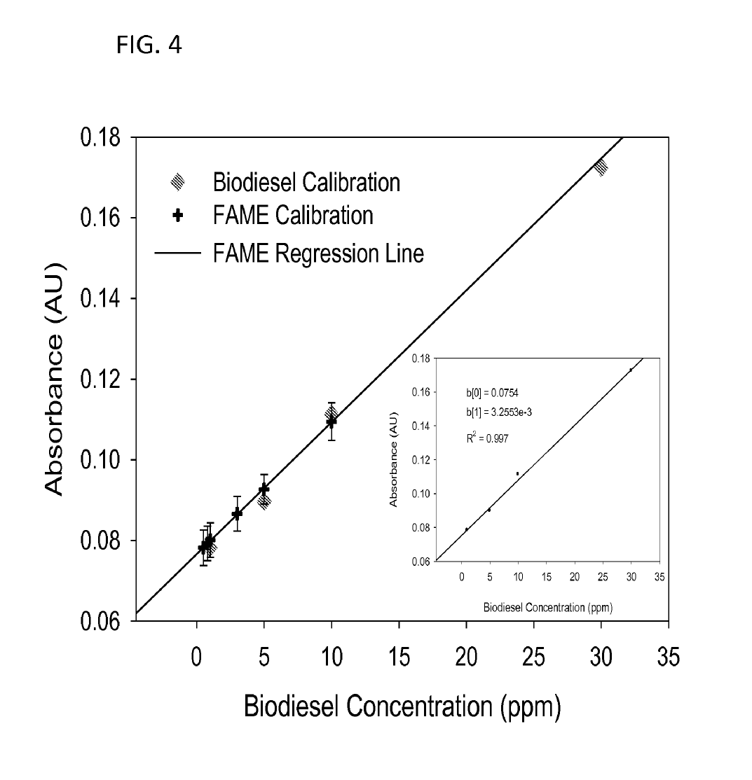

For low FAME concentrations of 0.5-30 ppm in diesel, the absorbance at 500 nm shows a linear increase (FIG. 4) both with standards and biodiesel samples made from a commercial biodiesel, B20 (20% biodiesel in diesel, also known as petrodiesel). The standards were first measured to establish the calibration plot in FIG. 4 with R²=0.999. The limit of detection (3σ) for the sensor was found to be 0.22 ppm and the limit of quantification 0.73 ppm, indicating high sensitivity for FAME detection. The response of the real biodiesel samples, prepared from a serial dilution of B20, is also shown in FIG. 4. The biodiesel calibration is overlaid onto the calibration plot to demonstrate that the results fall within the standard error of the FAME calibration curve (FIG. 4). For example, a 30 ppm biodiesel sample was evaluated using the FAME sensor. Using its absorbance value at 500 nm and the calibration plot gave the concentration of 29.30 ppm FAME with an accuracy of 97.7% (or error of 2.3%). The results here verify the validity of the calibration plot established by the standards. The tests with the commercial biodiesel demonstrate the reliability and application potentials of the new FAME sensor. The color change from blue to pink is evident with the naked eye. The FAME sensor may thus also serve as a qualitative, disposable one.

The sensor response to 20 mL of FAME, ranging from 0.5-30 ppm, is less than 30 min. Two steps have been taken to allow faster diffusion of FAME into the sensor film and reduce the response time. First, the mixture of ethyl cellulose and NBC dye can be spin-coated at 2600 rpm (rounds per minute) to make a thin sensor film. Second, the freshly-made sensor film is subjected to a dynamic vacuum (<0.01 mmHg) for 1 hour to remove toluene and excess MeOH/EtOH in the film. After the vacuum treatment, the sensor remains blue, indicating that there is sufficient MeOH/EtOH around the NBC dye molecules in the sensor. The response time is, however, reduced from ca. 60 min (without the vacuum treatment) to <30 min.

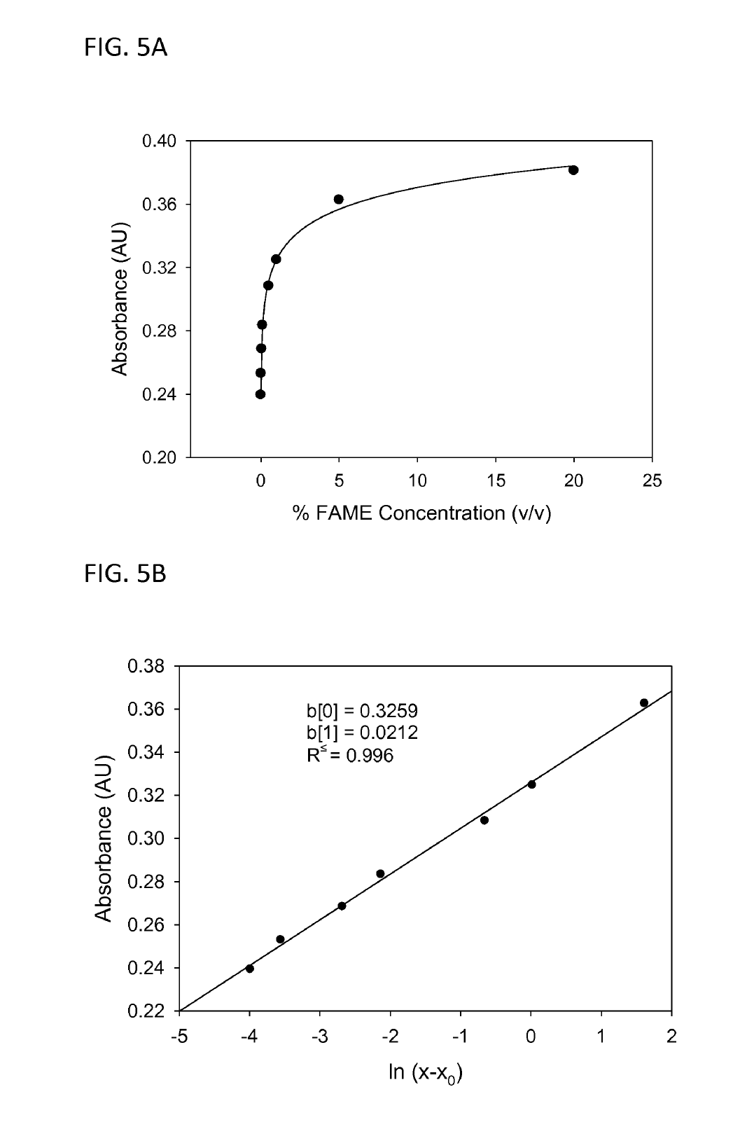

At concentrations higher than 30 ppm, the sensor begins to reveal a saturation effect, showing that a logarithmic curve function may be used. Plots involving 0 and 100-50,000 ppm (5% v/v) FAME standards are shown in FIGS. 5A and 5B. The absorbance A vs. In (x−x₀) (x is concentration of FAME, x₀ is given through the logarithmic fitting) gives a linear line with R²=0.996. At concentrations higher than 1,000 ppm, the sensor response time is less than 5 min.

### Materials

All solvents were obtained from Fischer Scientific and used as received. Ethyl cellulose (49% ethoxy content) was obtained from MP biomedicals and Nile Blue Chloride (NBC, 85%) was obtained from Sigma-Aldrich. Methyl hexanoate (98%) was obtained from Eastman, and methyl myristate (>98%) methyl oleate (>96%) methyl behenate (>90%) were obtained from Fischer Scientific and used as received. These FAME were used for make standard solutions in kerosene. Biodiesel (B20) containing 20% v/v FAME was obtained from a commercial source.

### Sensor Fabrication and Analyte Exposure

Standard microscope slides (Corning) were cut to 1 cm² squares and used as the sensor substrate. The glass squares were washed in a piranha solution (concentrated H₂SO₄ and 30% H₂O₂ in 3:1 ratio) for 30 min, followed by washes with acetone, methanol, ethanol, rinsed with deionized water, and allowed to dry in the oven before use. N-type [100] silicon wafers were similarly cleaned for deposition of thin film sensors that were then used for characterization by scanning electron microscopy (SEM).

Ethyl cellulose (˜0.750 g) was dissolved in a 1:1 mixture of toluene and ethanol and sonicated for approximately 4 hours to ensure that the ethyl cellulose was completely dissolved in solution. The result was a viscous ethyl cellulose solution (7.5% wt EC). NBC (˜1 mg) and methanol (350 μL) were added to 1.10 g of ethyl cellulose solution with stirring. This mixture was allowed to stand and cure for several days in a capped vial prior to use. After curing, the mixture was pipetted onto a clean glass slide and drawn to the edges of the glass with a plastic pipette tip. The slide was then spun at ˜2600 rpm (revolutions per minute) for approximately 1 min in a custom built spin-coater. After spin-coating, a freshly made thin film sensors with a distinct blue color were placed in a Schlenk tube and pumped at 0.01 mmHg vacuum for 1 hour. They were then stored prior to use.

The FAME mixture was made by combining methyl hexanoate, methyl myristate, methyl oleate, and methyl behenate in an evenly distributed 1:1:1:1 ratio. This mixture provides methyl esters with varying chain lengths (C6-C23) with methyl oleate offering a CH═CH bond in its chain. Different concentrations of the FAME mixture were made by diluting the mixture to the appropriate concentration with diesel. Sensors were submerged into their respective vials containing 20 mL of varying diesel/FAME concentrations while the solution was stirring. After satisfactory analysis time, the sensors were taken out of their vials and analyzed using a UV-Vis spectrometer.

The sensor response to 20 mL of FAME, ranging from 0.5-30 ppm, is less than 30 min. To achieve this response time, two steps have been taken to allow faster diffusion of FAME into the sensor film and reduce the response time. First, the mixture of ethyl cellulose and NBC dye needs to be spin-coated at 2600 rpm (revolutions per minute) to make a thin sensor film. Second, the freshly-made sensor film is subjected to a dynamic vacuum (<0.01 mmHg) for 1 hour to remove toluene and excess MeOH/EtOH in the film. After the vacuum treatment, the sensor remains blue, indicating that there is sufficient MeOH/EtOH around the NBC dye molecules in the sensor. These steps reduced the response time from originally over 1 hour (without the vacuum treatment) to <30 min.

### Instrumentation

An Agilent 8453 UV-Vis spectrometer using two light sources, a deuterium and tungsten lamp, was used to acquire absorbance spectra of the sensing films. A quartz cuvette with a 1 mm pathlength was utilized to hold the sensor in place. Spectra were recorded in the range from 190 cm⁻¹ to 1100 cm⁻¹. Peak deconvolution and baseline correction were achieved through Origin software. SEM images of the sensors before and after exposure to FAME were taken using a Leo 1525 Field Emission Scanning Electron Microscope.

Scanning electron microscopy (SEM) imaging was taken in order to characterize the surface of the FAME sensor before and after exposure to the FAME mixture. The images show “porous pitting” features consistent with the phase separation that occurs during the processing of the sensor with no discernible difference on the surface of the sensor before or after exposure. The porosity and polar features of the cellulose film allow FAME to preconcentrate and diffuse into the sensor, causing a color change.

### Stability Tests

Experiments were performed to test the stability of the FAME sensor over different periods of time. Extra sensors not used in previous experiments were kept in open air and then tested four months later against FAME sensors made a week ago and freshly made sensors made one day ago. These sensors were each exposed to real world samples of 1% biodiesel and examined in the UV-Vis. A side by side comparion in Table 1 shows that a freshly made sensor and a week old sensor have only a 0.3% error in absorbance at 502 nm while a sensor made 4 months ago left in open air had a 3% difference when compared to the freshly made sensor. This demonstrates that a sensor made several months ago is able to perform similarly to a freshly made FAME sensor that was fabricated only a day ago.

### Additional Biodiesel Studies

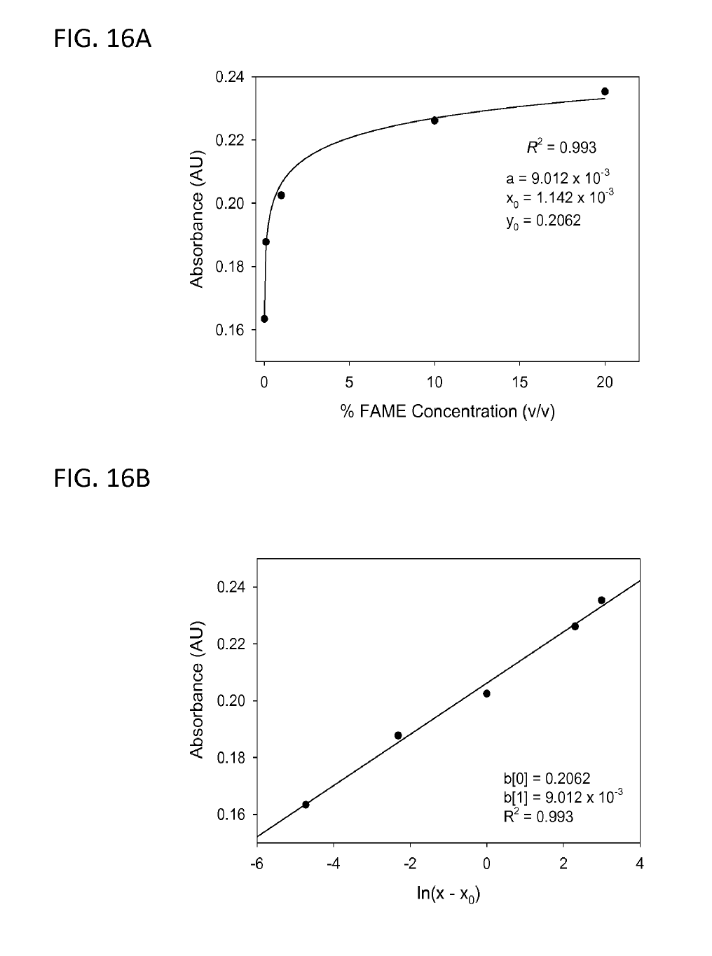

The FAME mixture used in previous experiments consisted of 4 different kinds of FAME ranging from C6-C23 which was used as a mimic for biodiesel standards. However, the FAME mixture that was used as standards is not entirely representative of the FAME composition found in biodiesel in the U.S and Europe, where methyl esters C16 and C18 dominate. Further tests have been performed using FAME senosr on comercially available biodiesel samples (containing C16 or C18 fatty acids with unsaturation sites) over the range of 100 ppm-20% FAME instead of a FAME mixture to serve as methyl ester standards. The data was analyzed using UV-Vis spectrometry and modelled using a lograthmic curve function (FIGS. 16A and 16B). The data is similar to previous experiments using FAME standards where the sensor undergoes a saturation effect. The absorbance (A) vs. In (x−x₀) of the biodiesel data gives a linear line with R²=0.993 (FIGS. 5A and 5B). These studies using biodiesel give a better representation of the target analyte and the detection of FAME. This study along with previous studies shows that this FAME sensor can detect FAME at a full range from 1 ppm to 20%.

### Example 2

The following dyes for biodiesel (fatty acid methyl esters or FAME) detection. The results here and earlier results using Nile Blue Chloride demonstrate that a solvatochromic compound (dye), dissolved in a solvent and placed in a thin film, can be used as an optical sensor for biodiesel (FAME).

1. Brilliant Cresyl Blue (BCB)

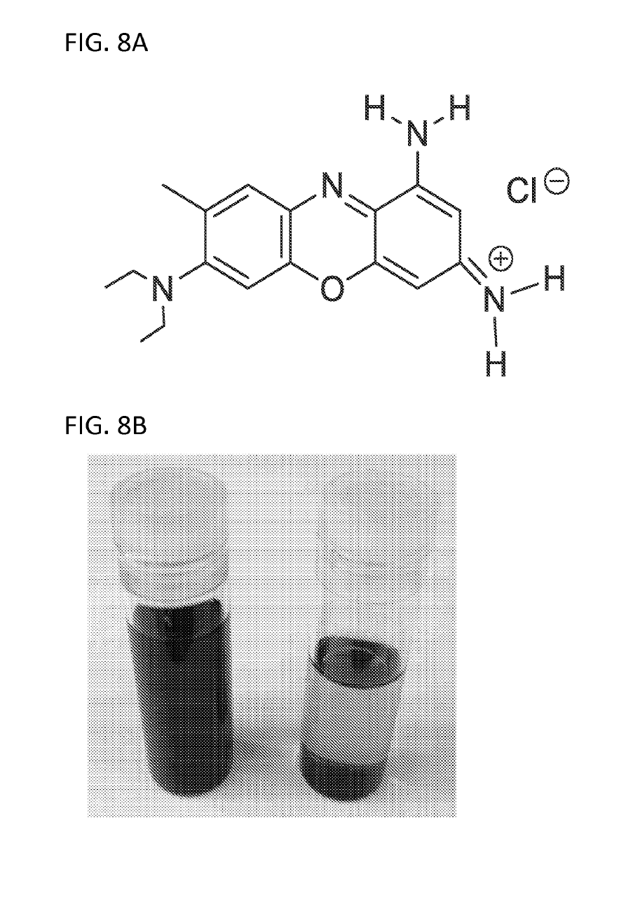

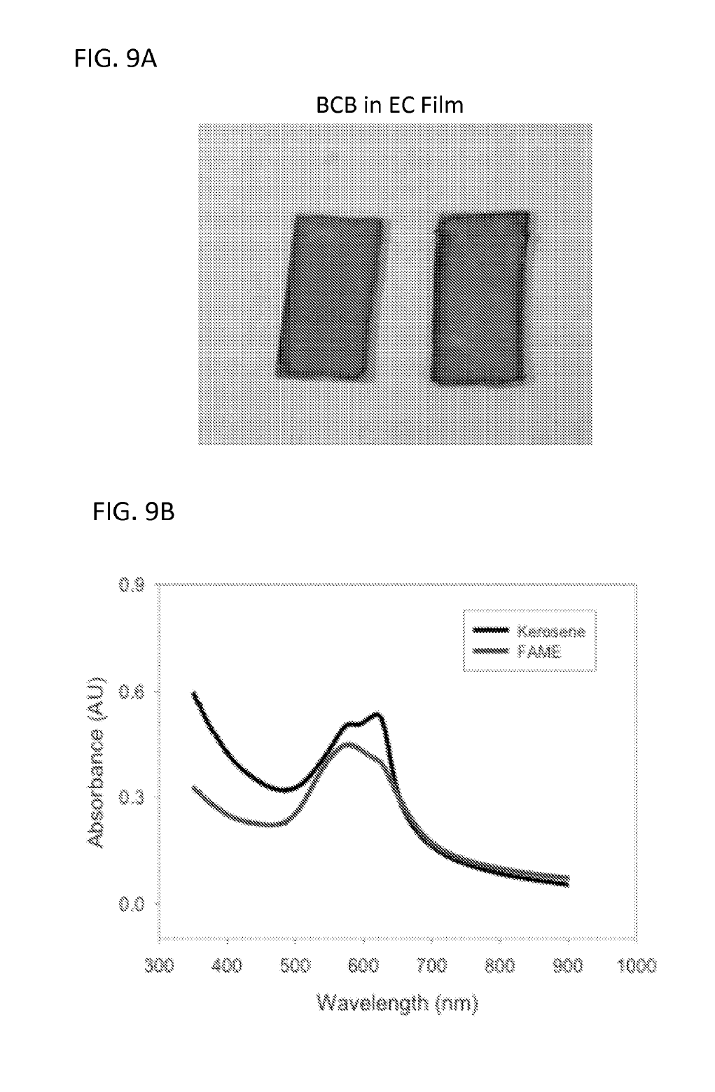

In solution phase, BCB turns to a dark blue color in methanol and in FAME it turns to a light purple (FIGS. 8A and 8B). When this dye is encapsulated into an EC thin film, it produces a blue sensor. The sensor was dipped into a 20% solution of FAME and the sensor's color changed from blue to purple (FIG. 9A). Testing the BCB film in the UV-Vis spectrometer shows that there are two noticeable peaks at 585 and 625 nm when the sensor is not exposed to FAME. However, after exposure to FAME, the sensor's spectrum changes and the peak at 620 nm disappears while the peak at 585 remains (FIG. 9B). Therefore, analyzing the peak disappearance at 620 nm for the BCB film may be used as a sensing mechanism for FAME detection.

2. Resorufin

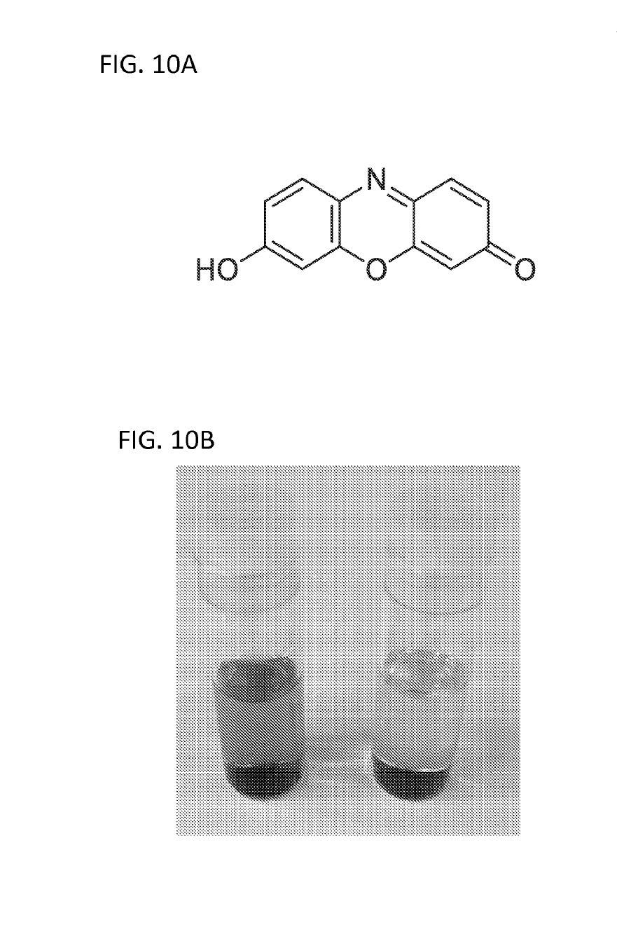

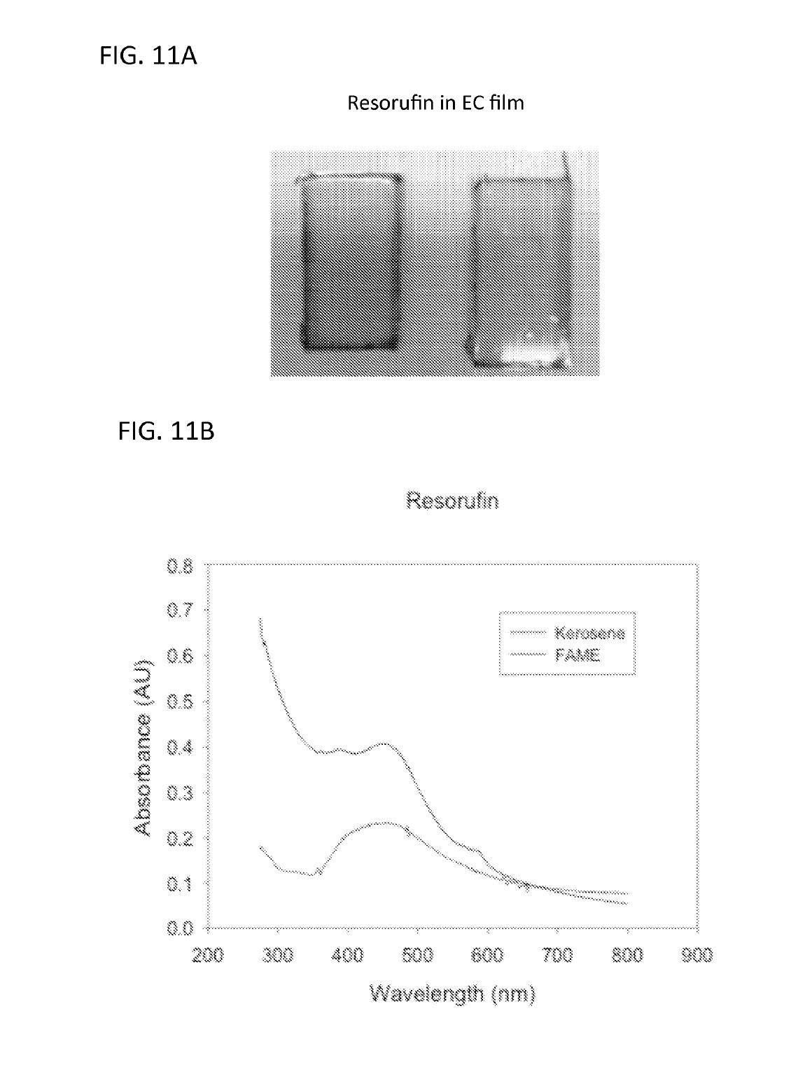

In methanol, Resorufin turns to a red/orange color but in FAME turns to a distinct yellow color (FIGS. 10A and 10B). This dye was encapsulated into a thin film using an EC solution and tested by exposing it to 20% FAME solution. When the film was exposed to FAME, it went from an orange color to a lighter orange color (FIG. 11A). This may be due to film decomposition. When the films were analyzed under UV-Vis spectrometry, it found that there was a distinct peak at 480-485 with no FAME exposure but the peak broadened greatly from 400 to 500 nm after exposed to 20% FAME (FIG. 11B).

3. Rhodamine 6G (R6G)

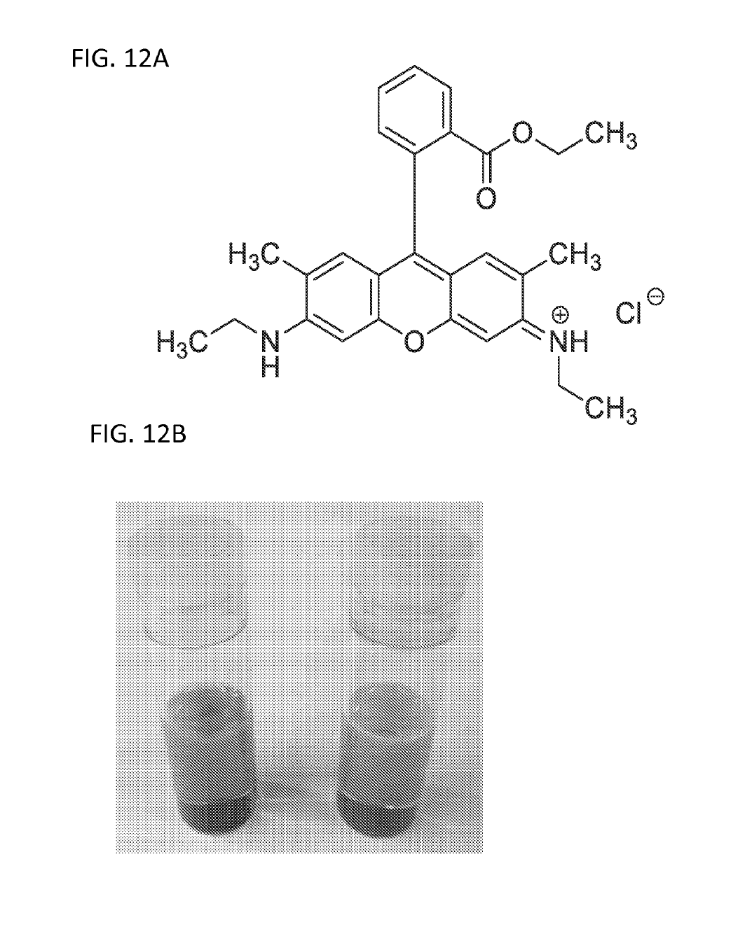

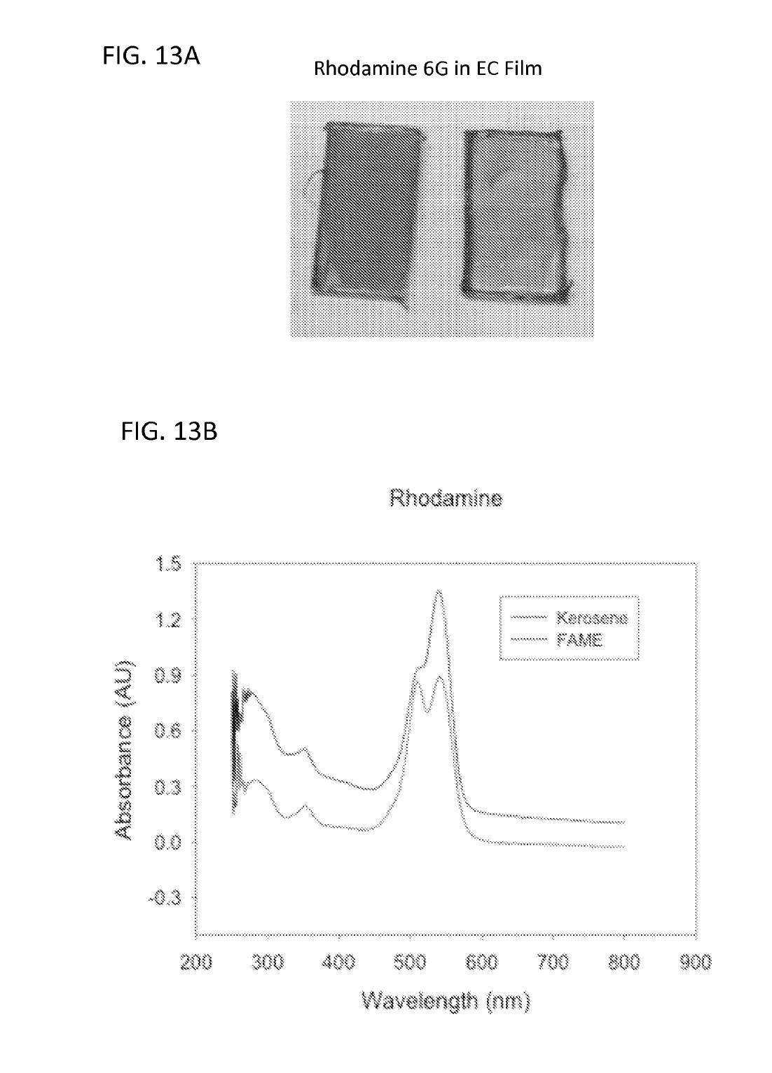

R6G is a very fluorescent dye and turns to a fluorescent pink color when dissolved in methanol and displays a distinct orange color when dissolved in FAME (FIGS. 12A and 12B). After encapsulated R6G into a thin EC film, it gave a pink sensor which was exposed to a 20% FAME solution. The result was a much lighter pink color (FIG. 13A). However, UV-Vis spectra show that there is a distinct peak at 560 nm and a slight shoulder at 520 nm when the sensor has not been exposed to FAME but after exposure to FAME, there is a depressed peak at 560 nm and a distinct appearance of a peak at 520 nm which was previously only a shoulder (FIG. 13B). This difference in spectra could be a useful as a detection mechanism for FAME.

4. Experimental Section

Ethyl cellulose was used as the polymer to encapsulate each dye by dissolving in a 1:1 mixture of toluene and ethanol and sonicating ca. 0.5 g of EC for approximately 4 hours to ensure that the EC was completely dissolved in solution. Then approximately 1 mg of each dye (BCB, Resorufin, R6G, and Ethidium bromide) was dissolved into 4 separate vials that contained 1.5 g of the EC solution. Each dye/EC mixture was stirred for 1 hour before use. After the dye was thoroughly mixed with the EC solution, each dye mixture was pipetted onto a glass substrate to form a thin film using the spin coating technique. The glass substrate was spun for 30 s with a spin rate of ca. 4000 rpm.

### Example 3

This example demonstrates that the methods disclosed herein can be applied to the detection of a polar compound in non-polar solution, such as a fuel solution.

### Experimental Section

Ethyl cellulose (EC) was dissolved in 0.85 g of THF (15 mL) by sonicating the mixture until the polymer was completely dissolved. Nile Blue Chloride (NBC, 3-4 mg) was added to 2 g of the EC-THF mixture. A stir bar was added and the mixture was stirred for ˜2 h.

Glass slides of 2×1 cm² were cut and placed onto the center of a spin coater using double sided tape. The NBC-EC-THF mixture (50 μL) was pipet onto the glass slide. After 15 s, the solution was spin-coated for 30 s at 2620 rpm (rounds per minute).

Phenol was dissolved in kerosene to give solutions with concentrations between 100 to 50.00 ppm. The sensors were placed in 20 mL of the solutions. Solutions of aniline, pyrrole, and carbazole were similarly prepared in kerosene.

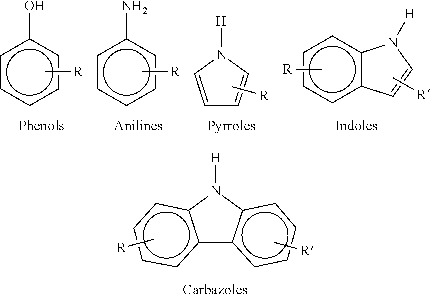

### Results and Discussion

### Detection of Phenol by the Optical Sensor

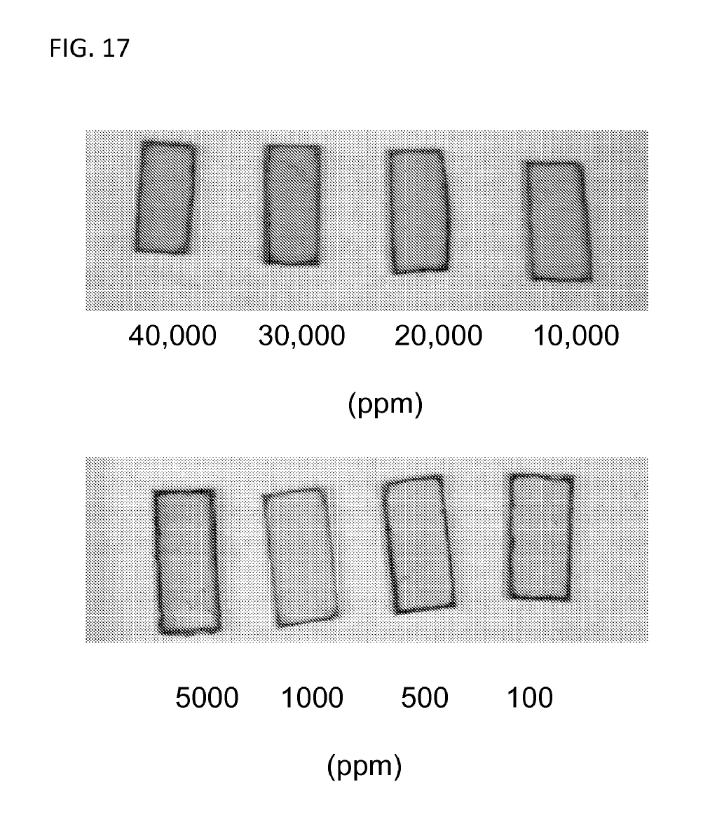

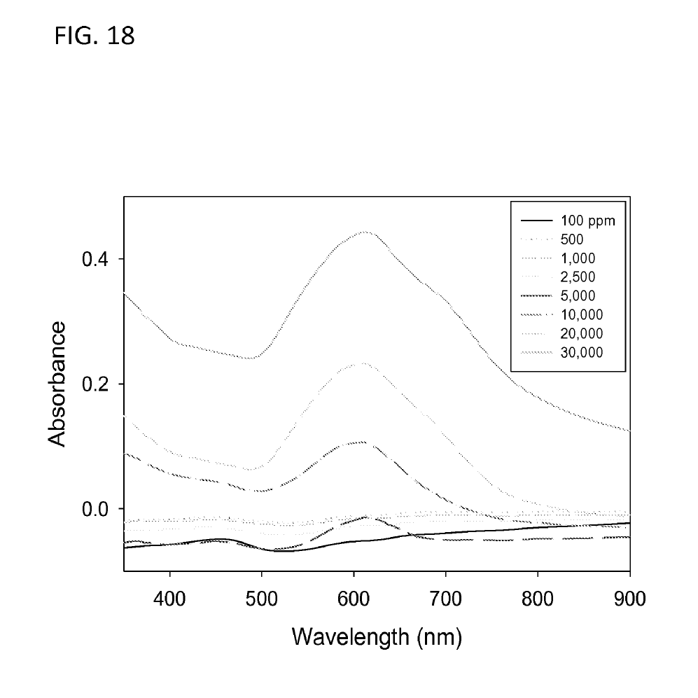

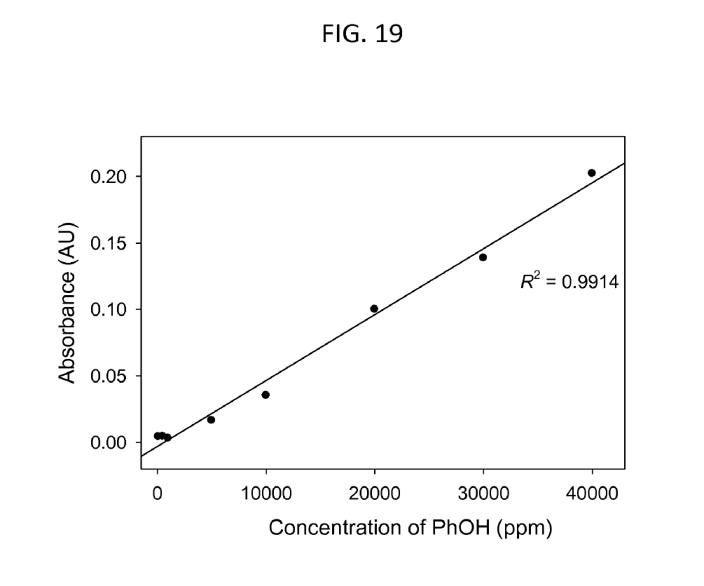

Fresh sensor films prepared in EC-THF thin films show light purple color. Photos of sensors after exposure to PhOH solutions are given in FIG. 17. Visible spectra of the sensors are given in FIG. 18. Calibration of the sensor response to PhOH solutions is given in FIG. 19. The detection and quantification limits of the sensor to PhOH were found to be 4.7 and 15.7 ppm, respectively in the conditions tested.

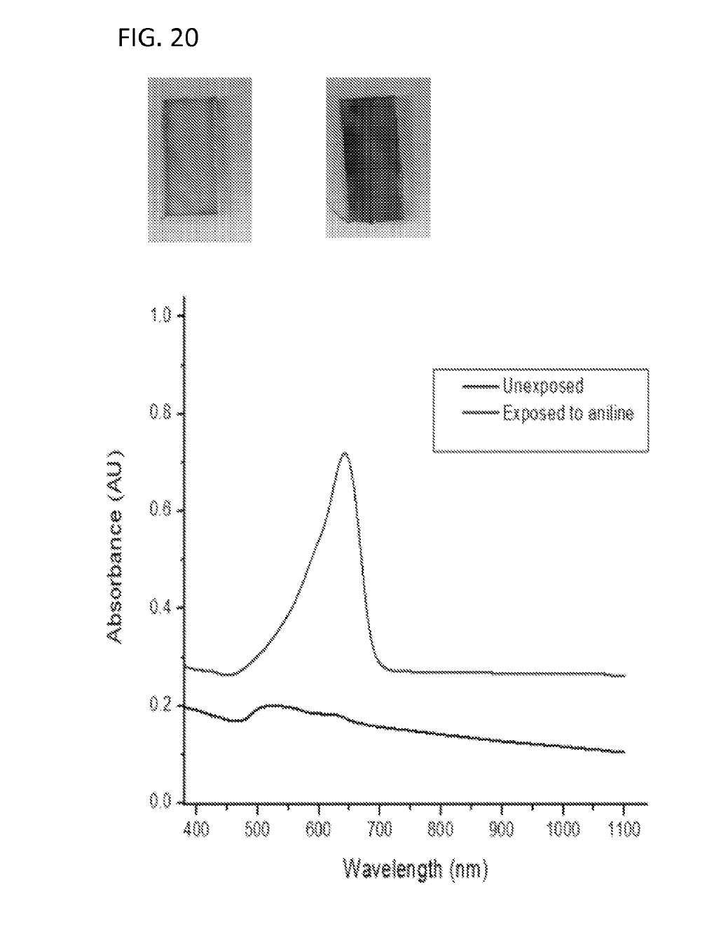

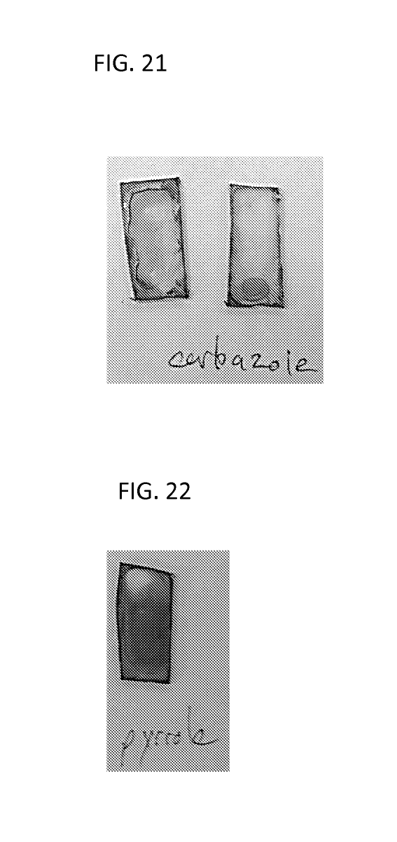

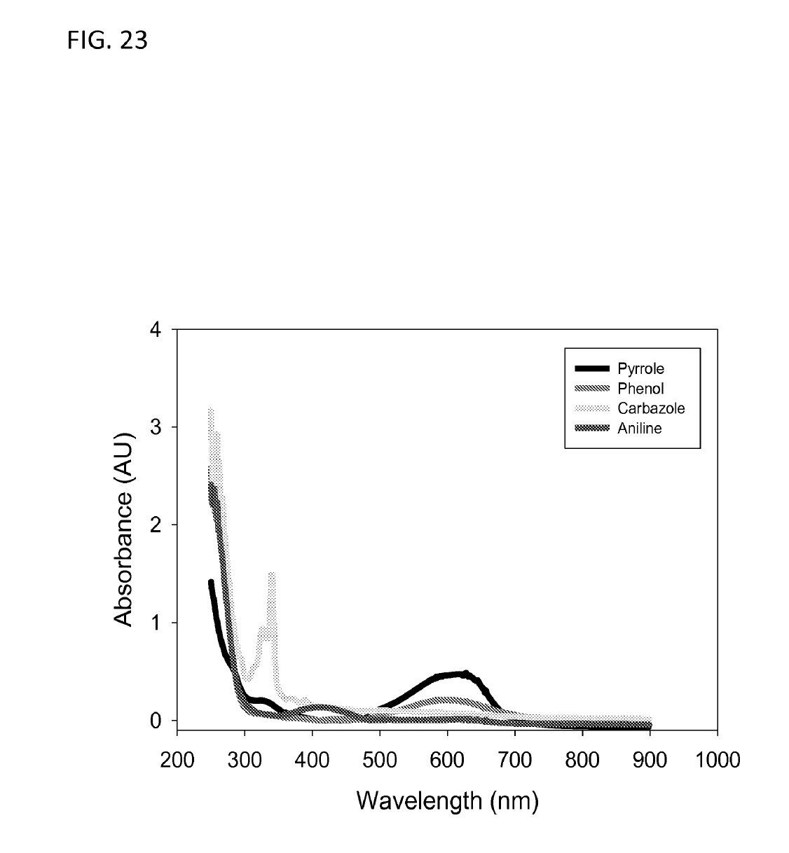

Responses of the optical sensor to N—H containing polar compounds were measured qualitatively. The results are given in FIGS. 20-22.

The results indicate that the sensors we have developed, dissolving solvatochromatic dyes in thin films, are good to detect trace polar compounds in fuels by ultraviolet-visible spectroscopy.

1. A method of detecting a polar compound in a sample, comprising:
contacting the sample with a solvatochromic compound, wherein the solvatochromic compound undergoes a shift in spectral absorbance when in contact with the polar compound in the sample as compared to the absorbance of the solvatochromic compound in a non-polar solvent; and
detecting a change in spectral absorbance of the solvatochromic compound, thereby detecting the polar compound in the sample.

2. The method of claim 1, wherein the polar compound comprises an ester, an alcohol, a carboxylic acid, an amine, an amide, a pyrrole, an indole, a carbazole, a pyridine, a quinolone, a sulfur-containing compound such as a thiol, thioether, a thiophenol, a ketone, an aldehyde, an ether, an acyl halide, an alkyl halide, cyclothiazide, 1,2-dithiane, benzothiazole, 2-benzothiophene, dihydro-5,6-dimethyl for a combination thereof.

3. The method of claim 2, wherein the polar compound comprises a fatty acid methyl ester (FAME).

4. The method of claim 3, wherein the fatty acid methyl ester is from biofuel.

5. The method of claim 4, wherein the biofuel is biodiesel.

6. The method of claim 2, wherein the alcohol comprises a phenol or a substituted derivative thereof.

7. The method of claim 2, wherein the amine comprises an aniline, pyrrole, indole, carbazole, prymadine, a substituted derivatives thereof, or a combination thereof.

8. The method of claim 1, wherein the polar compound is a product of pyrolysis of biomass.

9. The method of claim 8, wherein the biomass comprises cellulose, lignin or a combination thereof.

10. The method of claim 1, further comprising comparing the detected spectral absorbance to a control.

11. The method of claim 10, wherein the control is one or more values indicative of a known concentration of a known polar compound.

12. The method of claim 10, wherein the control is one of more control samples with a known concentration of a known polar compound.

13. The method of claim 10, wherein the control is a spectral absorbance of one of more control samples with a known concentration of a known polar compound.

14. The method of claim 10, wherein the control is a calibration curve for a known polar compound.

15. The method of claim 11, wherein the known polar compound comprises an ester, an alcohol, a carboxylic acid, an amine, an amide, a pyrrole, an indole, a carbazole, a pyridine, a quinolone, a sulfur-containing compound such as a thiol, a thioether, a thiophenol, a ketone, an aldehyde, an ether, an acyl halide, an alkyl halide, or a combination thereof.

16. The method of claim 1, further comprising quantifying the amount of the polar compound in the sample.

17. The method of claim 1, wherein the sample comprises a fuel sample.

18. The method of claim 17, wherein the fuel sample comprises a diesel fuel sample.

19. The method of claim 18, wherein the biofuel comprises biodiesel.

20. The method of claim 17, wherein the fuel sample comprises a biofuel sample.

21. The method of claim 20, wherein the biofuel sample comprises a pyrolysis product of biomass.

22. The method of claim 21, wherein the biomass comprises cellulose, lignin or a combination thereof.

23. The method of claim 1, wherein the solvatochromic compound comprises a lipophilic solvatochromic compound.

24. The method of claim 1, wherein the solvatochromic compound comprises one or more of Nile Blue (5-amino-9-(diethylamino)benzo[a]phenoxazin-7-ium chloride), brilliant cresyl blue, resorufin (7-Hydroxy-3H-phenoxazin-3-one 10-oxide), or rhodamine 6g (r6g).

25. The method of claim 1, wherein the solvatochromic compound is immobilized in or on a solid or semi solid material.

26. The method of claim 25, wherein the solid or semi solid material comprises a polymer.

27. The method of claim 25, wherein the solid or semi solid material comprises ethyl cellulose, silica sol-gels, or organically modified sol-gels.

28. The method of claim 25, wherein the solid or semi solid material comprises a membrane or thin film.

29. The method of claim 1, wherein the change in absorbance result in is change in the perceived color of the solvatochromic compound.

30. The method of claim 1, wherein the method detects the amount of biodiesel present in a diesel fuel sample.

31. The method of claim 1, wherein the method detects the amount of polar compounds present in a biofuel sample.

32. The method of claim 1, wherein spectral change is detected using ultraviolet-visible spectroscopy.
